(19) United States
(12) Patent Application Publication
(10) Pub. No.: US 2021/0008274 A1
(43) Pub. Date: Jan. 14, 2021

(54) DRUG SOLUTION ADMINISTRATION DEVICE AND CONTROL METHOD FOR DRUG SOLUTION ADMINISTRATION DEVICE

(71) Applicant: TERUMO KABUSHIKI KAISHA, Tokyo (JP)

(72) Inventors: Akira KONDO, Kanagawa (JP); Yusuke YAKUSHIJI, Kanagawa (JP)

(73) Assignee: TERUMO KABUSHIKI KAISHA, Tokyo (JP)

(21) Appl. No.: 17/037,181

(22) Filed: Sep. 29, 2020

Related U.S. Application Data
(63) Continuation of application No. PCT/JP2019/013893, filed on Mar. 28, 2019.

(30) Foreign Application Priority Data
Mar. 30, 2018 (JP) 2018-067431

(51) Int. Cl. A61M 5/145; A61M 5/168; A61M 5/315

(52) U.S. Cl. CPC A61M 5/1452; A61M 5/168; A61M 5/14248; A61M 2205/8206; A61M 5/3157

(57) ABSTRACT

A drug solution administration device is able to prevent liquid supply of a drug solution from ending in a state where some of the drug solution remains in a drug solution container. The drug solution administration device includes a drug solution container filled with a drug solution and having a distal end opening, a housing that holds the drug solution container, a plunger that pushes out the drug solution in the drug solution container, a drive mechanism that advances the pusher toward the distal end opening of the drug solution container, a detection unit that detects a detection target part of the plunger to detect liquid supply completion of the drug solution based on a detection result, and a control unit that controls an operation of the drive mechanism. The control unit further advances the pusher after the detection unit detects the liquid supply completion.

### CROSS-REFERENCES TO RELATED APPLICATIONS

This application is a continuation of International Application No. PCT/JP2019/013893 filed on Mar. 28, 2019, which claims priority to Japanese Patent Application No. 2018-067431 filed on Mar. 30, 2018, the entire content of both of which is incorporated herein by reference.

### FIELD OF THE INVENTION

The present invention generally relates to a drug solution administration device and system, and a control method for a drug solution administration device and system.

### BACKGROUND DISCUSSION

Conventionally, there is known a syringe pump drug solution administration device that administers a drug solution filled in a drug solution container into a living body by a pushing action of a pusher. This type of drug solution administration device includes a cylindrical drug solution container and a pusher mechanism that pushes out the drug solution in the drug solution container (see, for example, Japanese Patent Application Publication No. 09-294807). The drug solution container and the pusher mechanism are accommodated in a housing and supported by the housing.

### SUMMARY

In the drug solution administration device as described above, completion of a drug solution filled in the drug solution container is performed, for example, by detecting, with a detection switch installed on an electric board or the like, that the pusher mechanism has moved forward by a predetermined distance. When the detection switch comes into contact with the pusher mechanism and the contact is detected, a control unit mounted on the drug solution administration device determines that the drug solution has run out (i.e., has been fully administered), and stops a drive mechanism that moves the pusher forward. After that, the control unit notifies a user that the liquid supply is completed.

In the drug solution administration device, if there is a deviation in installation accuracy of the detection switch, the pusher mechanism, the drug solution container, and the like, or dimensional accuracy of the pusher mechanism, the drug solution container, and the like, it becomes difficult to accurately detect a movement amount of the pusher mechanism by the detection switch. For example, if there is a deviation in the installation accuracy and the dimensional accuracy as described above, there is a possibility that the detection switch may detect “liquid supply completion (zero residual liquid)” even though drug solution remains in the drug solution container. As a result, the drug solution administration device can no longer administer an amount of the drug solution that should be originally administered to the living body.

The drug solution administration device and control method for a drug solution administration device disclosed here can prevent liquid supply of a drug solution from ending in a state where a drug solution remains in a drug solution container.

The drug solution administration device described here includes: a drug solution container filled with a drug solution and having a distal end opening at a distal end of the drug solution container; a housing in which the drug solution container is positioned; a plunger that pushes out the drug solution in the drug solution container so that the drug solution is discharged out of the distal end opening at the distal end of the drug solution container, the plunger including a detection target part; a drive mechanism operatively connected to the plunger to advance the plunger toward the distal end opening of the drug solution container; and a detection unit that detects the detection target part of the plunger and detects liquid supply completion of the drug solution when the detection unit detects the detection target part of the plunger. In addition a control unit is operatively connected to the drive mechanism and controls operation of the drive mechanism based on detection results received from the detection unit. The control unit is configured to operate the drive mechanism to further advance the plunger toward the distal end opening of the drug solution container after the detection unit detects the liquid supply completion.

According to another aspect, a drug solution administration system comprises: a housing; a drug solution container filled with a drug solution and positioned in the housing; a plunger positioned in the drug solution container so that the drug solution in the drug solution container is between the discharge port of the drug solution container and a distal end of the plunger; and a needle configured to puncture a living body. The plunger is movable relative to the drug solution container to advance toward the distal end of the drug solution container to discharge the drug solution out of the drug solution container through the distal end of the drug solution container. The plunger includes a detection target part that moves together with the plunger. The needle is in fluid communication with the distal end of the drug solution container so that the drug solution discharged out of the drug solution container through the distal end of the drug solution container passes through the needle. A motor is operatively connected to the plunger to advance the plunger toward the distal end of the drug solution container to discharge the drug solution out of the drug solution container through the distal end. A sensor that detects the detection target part of the plunger as the plunger advances toward the discharge port of the drug solution container to identify a liquid supply completion state of the drug solution administration system, with the sensor emitting a signal when the sensor detects the detection target part of the plunger. A control unit that receives the signal from the sensor when the sensor detects the detection target part of the plunger and that is operatively connected to the motor to control operation of the motor. The control unit is configured to operate the motor after the sensor detects the detection target part of the plunger so that there is further advancement of the plunger toward the distal end of the drug solution container after the sensor detects the detection target part of the plunger.

Another aspect involves a control method for controlling movement of a plunger positioned in a drug solution container that is filled with a drug solution so that the drug solution is between the plunger and a distal end of the drug solution container. The drug solution container and the plunger are positioned in a housing, the drug solution container includes a distal end opening at the distal end of the drug solution container, plunger includes a detection target part that moves together with the plunger, and a detection unit detects the detection target part of the plunger. The control method comprises: advancing the plunger inside the drug solution container toward the distal end opening of the drug solution container to discharge the drug solution out of the drug solution container through the distal end opening of the drug solution container; detecting the detection target part through operation of the detection unit to determine that a liquid supply completion state has been reached; and further advancing the plunger toward the distal end opening of the drug solution container after the detecting of the detection target part through operation of the detection unit.

According to the drug solution administration device and system, and the control method for the drug solution administration device and system disclosed here, it is possible to prevent liquid supply of the drug solution from ending in a state where the drug solution remains in the drug solution container.

### BRIEF DESCRIPTION OF THE DRAWINGS

FIG. 1 is a side view of a drug solution administration system according to an embodiment.

FIG. 2 is a view schematically showing a usage example of the drug solution administration system.

FIG. 3 is a schematic perspective view of a drug solution administration device.

FIG. 4 is a schematic perspective view of a chassis included in a housing, and each component member assembled to the chassis.

FIG. 5 is a block diagram schematically showing a control system of the drug solution administration device.

FIG. 6 is a flowchart schematically showing a control method for a drug solution administration device according to the embodiment.

FIG. 7 is a plan view of the drug solution administration device, showing a state before a pusher moves forward.

FIG. 8 is a plan view of the drug solution administration device, showing a state after the pusher moves forward.

FIG. 9 is a view showing a positional relationship between the pusher and a detection unit.

FIG. 10 is a plan view showing a contrast example.

FIG. 11 is a plan view showing a contrast example.

### DETAILED DESCRIPTION

Set forth below with reference to the accompanying drawings is a detailed description of an embodiment of a drug solution administration device and system, and a control method for a drug solution administration device and system representing examples of the inventive drug solution administration device, drug solution administration system and control method disclosed here. The following description does not limit the technical scope or meaning of terms used in the claims. Further, the dimensional ratios in the drawings are exaggerated for convenience of description, and may differ from the actual ratios.

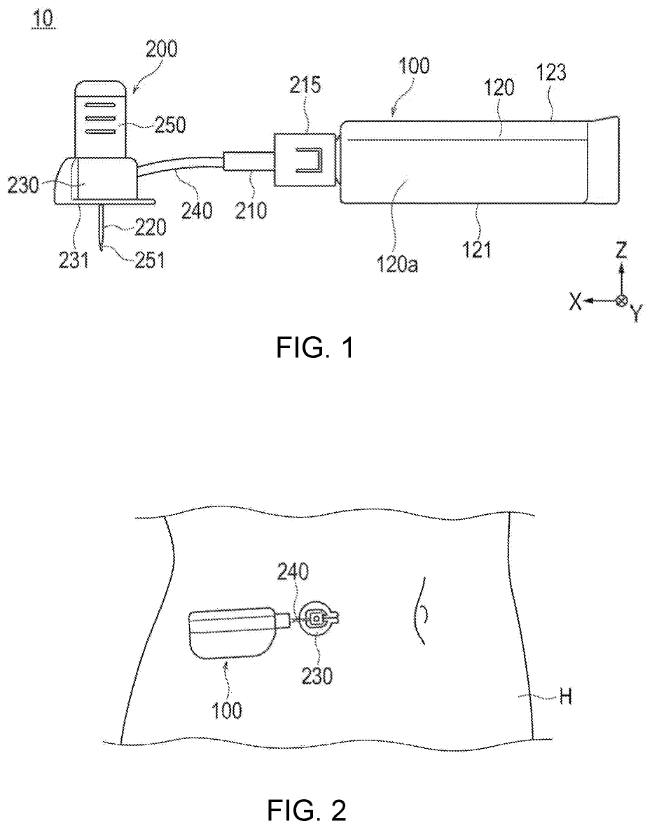

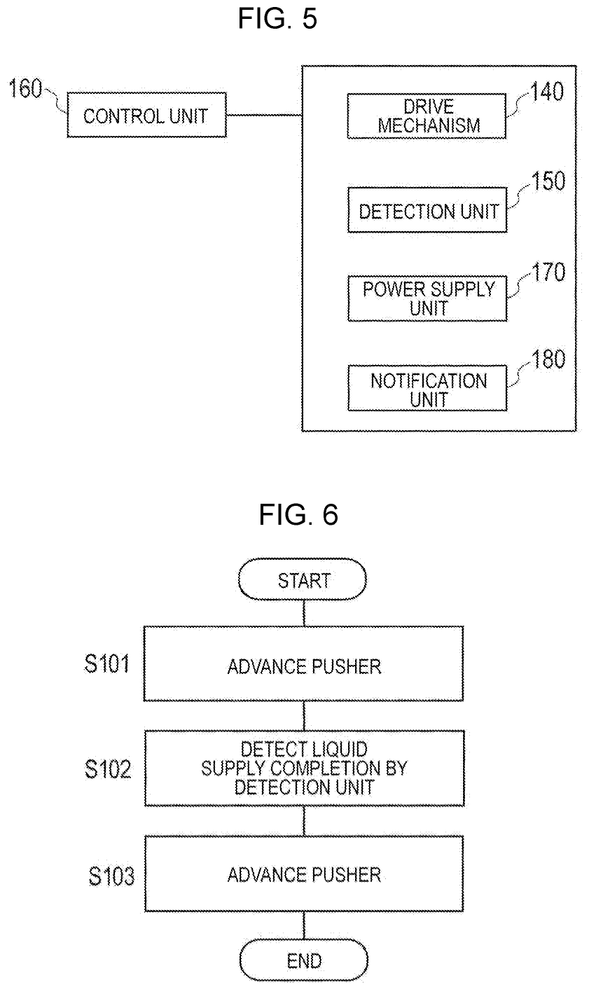

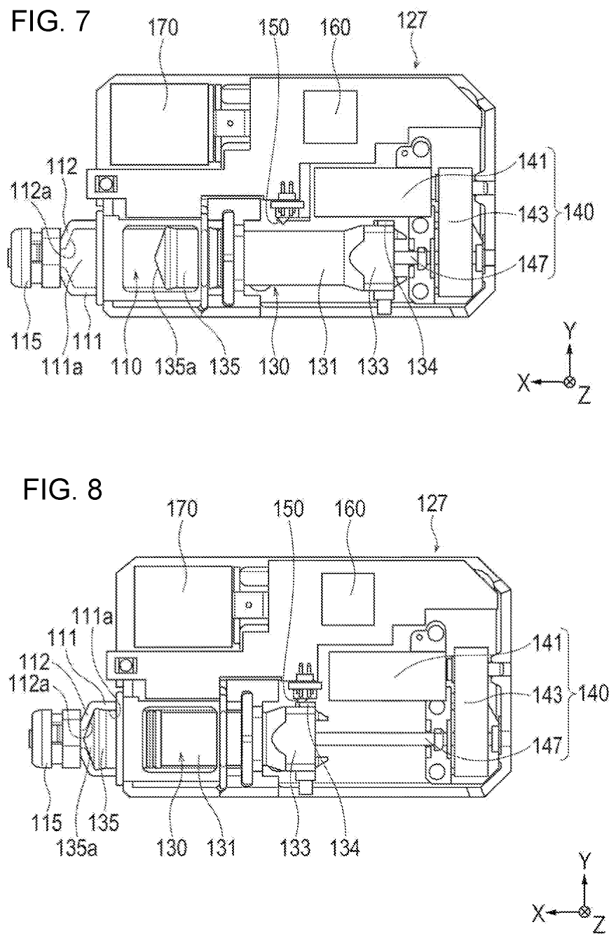

FIGS. 1 to 4 illustrate a drug solution administration system 10, a drug solution administration device 100, and an administration tool 200 according to one embodiment disclosed by way of example. FIG. 5 is a block diagram schematically showing a control system of a control unit 160, and FIG. 6 is a flowchart showing a control method for a drug solution administration device. Further, FIGS. 7 to 8 are views for explaining an action of the drug solution administration device 100 and a comparative example. Arrow X attached to each figure indicates a “longitudinal direction (a longitudinal direction of a drug solution container 110)” of the drug solution administration device 100, arrow Y indicates a “width direction (a depth direction)” of the drug solution administration device 100, and arrow Z indicates a “height direction” of the drug solution administration device 100.

(Drug Solution Administration System)

The drug solution administration system 10 is used to administer a drug solution into a living body. As shown in FIG. 1, the drug solution administration system 10 includes the drug solution administration device 100 and the administration tool 200.

As shown in FIG. 2, the drug solution administration device 100 and the administration tool 200 are configured as a patch-type device or tool that is used by being attached to a body surface (skin) H of a user. A body part of the user to be attached with the drug solution administration device 100 and the administration tool 200 is not particularly limited, but is, for example, an abdomen or a thigh.

The drug solution administration system 10 can continuously administer, for example, a drug solution (not shown) filled in the drug solution container 110 included in the drug solution administration device 100, into a living body over a relatively long time (for example, several minutes to several hours) through a pushing action of a pusher or plunger 130 described later. The drug solution administration system 10 may intermittently administer the drug solution into the living body.

(Drug Solution Administration Device)

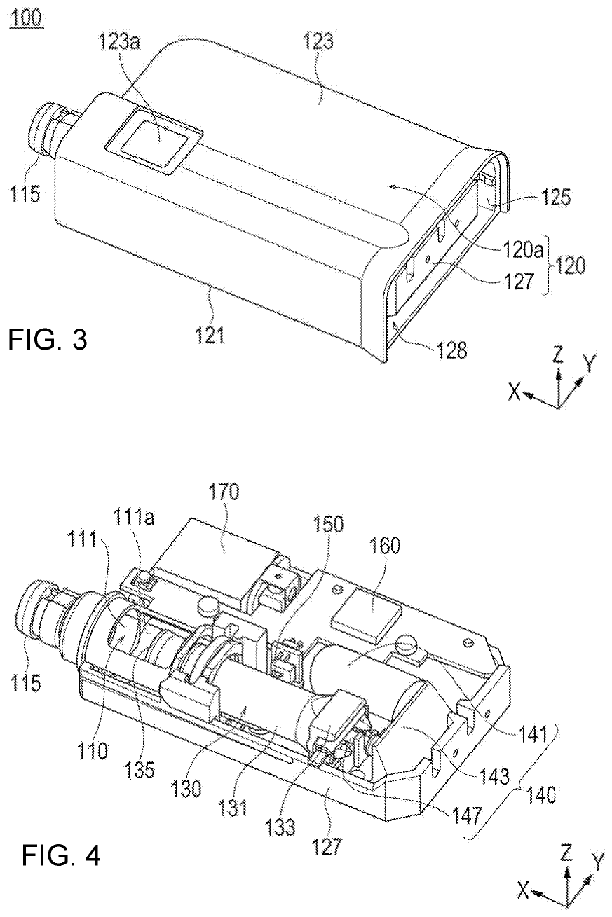

As shown in FIGS. 3, 4, 7, and 8, the drug solution administration device 100 has the drug solution container 110 including a cylindrical (barrel-shaped) main body 111 filled with a drug solution, a housing 120 that holds the drug solution container 110, the pusher or plunger 130 that pushes out or expels the drug solution in the drug solution container 110, a drive mechanism 140 that advances the pusher 130 toward a distal end opening of the drug solution container 110, a detection unit 150 that detects a detection target part 134 of the pusher 130 and detects liquid supply completion of the drug solution based on a detection result, and the control unit 160 that controls an operation of the drive mechanism.

As shown in FIGS. 3 and 4, the housing 120 has a box-shaped housing main body 120a in which an accommodation space 128 is internally formed, and a chassis (corresponding to a “supporting part”) 127 that is accommodated in the accommodation space 128 of the housing main body 120a and can be fixed to the housing main body 120a.

As shown in FIG. 3, on an upper surface 123 of the housing main body 120a, there is formed a window 123a that enables visual recognition of an inside of the accommodation space 128 from an outside of the housing 120. The window 123a is formed by providing a transparent or semitransparent portion on a part of the housing main body 120a.

On a base end side in the longitudinal direction of the housing main body 120a, there is formed a base end opening 125 for inserting the chassis 127 into the accommodation space 128 of the housing main body 120a. The base end opening 125 of the housing main body 120a is closed by a lid member (not shown), with the chassis 127 accommodated in the accommodation space 128.

A bottom surface 121 of the housing main body 120a is provided with a sheet-shaped sticking part that can be stuck or adhered to the body surface H of the user. In an initial state before attaching the drug solution administration device 100 to the user, a releasable protective sheet is attached to a sticking or adhesive surface of the sticking part.

As shown in FIG. 4, the chassis 127 holds the drug solution container 110, the pusher 130, the drive mechanism 140, the detection unit 150, the control unit 160, and a power supply unit 170.

The drug solution container 110 is a pre-filled drug solution container. Therefore, the drug solution is filled in advance in an inner cavity or interior 111a of the main body 111 of the drug solution container 110. Examples of the drug solution include a protein preparation, a narcotic analgesic, a diuretic, and the like.

A sealing member (not shown) is disposed at a distal end opening (a discharge port) at a distal end 112 of the drug solution container 110 to prevent leaking of the drug solution is arranged. As shown in FIG. 3, the distal end opening of the drug solution container 110 is arranged so as to externally project from and be positioned outside the housing main body 120a. Further, a mounting part 115, which is to be connected to a tube 240 (see FIG. 1) described later, is attached to a distal end portion of the drug solution container 110 projecting from the housing main body 120a.

A main body 131 of the pusher 130 is inserted into or positioned in the inner cavity 111a of the main body 111 of the drug solution container 110 (see FIGS. 4 and 7). At a distal end of the main body 131 of the pusher 130, a gasket 135 that is in slidable contact with an inner wall of the drug solution container 110 is arranged. A base end side of the gasket 135 is liquid-tightly sealed, by an outer peripheral portion of the gasket 135 being in liquid-tight contact with an inner peripheral surface of the main body 111 of the drug solution container 110.

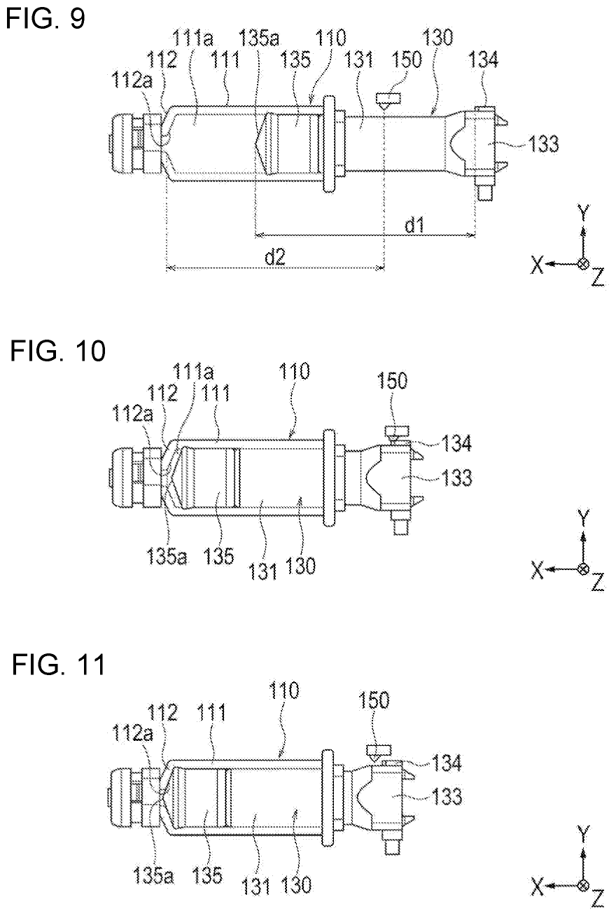

In the present embodiment, the gasket 135 is configured to be able to contract in an advancement direction (the longitudinal direction) of the pusher 130, when the pusher 130 advances in a state where the gasket 135 abuts against a distal-end inner wall 112a (see FIG. 9) of the drug solution container 110. The gasket 135 can be made by a flexible resin material such as, for example, a rubber material or an elastomer so as to be able to contract as described above.

As shown in FIG. 9, the gasket 135 has a tapered shape in which an outer diameter decreases toward a distal end side. Further, the shape of the gasket 135 is formed to be substantially the same as a shape of the distal-end inner wall 112a of the drug solution container 110.

As shown in FIG. 9, the detection target part 134 is provided at a base end of the pusher 130. The detection target part 134 is used to detect liquid supply completion of the drug solution by the drug solution administration device 100 (drug solution discharge or supply completion state). The method of detecting the liquid supply completion will be described later.

The control unit 160 controls a liquid supply operation of the drug solution of the drug solution administration device 100. The control unit 160 can be configured by, for example, a known microcomputer (an electronic circuit element) mounted with a CPU, a RAM, a ROM, and the like. The control unit 160 centrally executes operation control of the drive mechanism 140, the detection unit 150, the power supply unit 170, and a notification unit 180 (see FIG. 5).

As shown in FIG. 7, the detection unit 150 is arranged on the chassis 127. As shown in FIG. 8, when the detection target part 134 included in the pusher 130 comes into contact with the detection unit 150, the detection unit 150 detects liquid supply completion of the drug solution administration device 100. The detection unit 150 can be configured by, for example, a known contact sensor that emits a predetermined electric signal when the detection target part 134 comes into contact with the detection unit 150. The control unit 160 receives the electric signal from the detection target part 134, to acquire information regarding the liquid supply completion. The detection unit 150 is not particularly limited in a specific configuration and the like as long as it is possible to detect a position of the detection target part 134 of the pusher 130 when the pusher 130 advances by a predetermined amount.

The power supply unit 170 can be configured by, for example, a known battery or the like.

The notification unit 180 notifies the user of liquid supply completion. The notification unit 180 can be configured by, for example, a speaker or the like that emits voice or sound indicating that the liquid supply is completed. The notification unit 180 can be accommodated in the accommodation space 128 of the housing main body 120a.

As shown in FIG. 4, the drive mechanism 140 includes a motor 141 that receives a driving current from the power supply unit 170 to generate a rotational driving force, a reduction mechanism 143 including a gear or the like to transmit the rotational driving force of the motor 141, and a feed screw 147 connected to the reduction mechanism 143.

The feed screw 147 is connected to a base-end connecting part 133 arranged near the base end of the pusher 130. The feed screw 147 converts a rotary motion transmitted from the reduction mechanism 143 into a linear motion, and advances the pusher 130 in the longitudinal direction (the X direction). The pusher 130 pushes out the drug solution from the inner cavity 111a of the main body 111 of the drug solution container 110 to the tube 240 (see FIG. 1), by advancing toward the distal end side of the drug solution container 110.

Next, with reference to FIG. 9, a positional relationship between the detection unit 150 and the detection target part 134 will be described. FIG. 9 shows the drug solution container 110 and the pusher 130 in a state before administering the drug solution.

In the present embodiment, the detection unit 150 is arranged so as to detect liquid supply completion before a distal end 135a of the pusher 130 abuts against the distal-end inner wall 112a of the drug solution container 110. More specifically, as shown in FIG. 9, a first distance (d1) from the distal end 135a of the pusher 130 to the detection target part 134 is shorter than a second distance (d2) from the distal-end inner wall 112a of the drug solution container 110 to a position where the detection unit 150 is arranged. Therefore, when the pusher 130 advances in the forward direction (i.e., leftward direction in FIG. 9) in the drug solution container 110 to cause the detection target part 134 to come into contact with the detection unit 150, and the detection unit 150 detects liquid supply completion, there is formed a gap (space) in which the pusher 130 can further advance in the forward direction, between the distal-end inner wall 112a of the drug solution container 110 and the distal end 135a of the pusher 130.

Note that the distal end 135a of the pusher 130 means the distal end of the gasket 135 when the gasket 135 is arranged on the pusher 130 as in the present embodiment.

Next, a control method for the drug solution administration device 100 according to the present embodiment will be described. Hereinafter, operation control of the pusher 130 at a time of liquid supply of a drug solution will be described in detail.

As shown in FIG. 6, the control method for the drug solution administration device 100 has a step of advancing the pusher 130 in the drug solution container 110 (S101), a step of detecting liquid supply completion by the detection unit 150 detecting the detection target part 134 of the pusher 130 (S102), and a step of advancing the pusher 130 after liquid supply completion is detected by the detection unit 150 (S103).

FIG. 7 shows a state before the pusher 130 advances in the drug solution container 110. When the pusher 130 advances in the drug solution container 110 from the state shown in FIG. 7, and the detection target part 134 comes into contact with the detection unit 150 as shown in FIG. 8, the detection unit 150 detects the liquid supply completion.

In the control method according to the present embodiment, after the liquid supply completion is detected as described above, the pusher 130 is further advanced by a distance equal to or more than a distance until the distal end 135a of the pusher 130 abuts against the distal-end inner wall 112a of the drug solution container 110. This is for the following reason.

For example, in the drug solution administration device 100, if there is a deviation in installation accuracy of the detection unit 150, the pusher 130, the drug solution container 110, and the like, or dimensional accuracy of the pusher 130, the drug solution container 110, and the like, as shown in FIG. 10, the detection unit 150 detects liquid supply completion despite being in a state where drug solution remains in the drug solution container 110 (a state where the distal end 135a of the pusher 130 is not in contact with the distal-end inner wall 112a of the drug solution container 110). If the liquid supply by the drug solution administration device 100 ends as it is, it becomes impossible to administer an amount of the drug solution that should be originally administered to the living body. With respect to such a problem, the drug solution administration device 100 according to the present embodiment further advances the pusher 130 after the detection unit 150 detects the liquid supply completion. Therefore, the drug solution administration device 100 can preferably prevent occurrence of “remaining drug solution” due to a deviation or the like in installation accuracy or dimensional accuracy of each member. Particularly, in the present embodiment, it is possible to more reliably prevent the drug solution from remaining in the drug solution container 110 since the pusher 130 is advanced by a distance equal to or more than a distance until the distal end 135a of the pusher 130 abuts against the distal-end inner wall 112a of the drug solution container 110 after the liquid supply completion is detected.

Further, if there is a deviation or the like in installation accuracy or dimensional accuracy of each member, as shown in FIG. 11, there may also be a problem that the detection unit 150 does not detect the liquid supply completion although the pusher 130 has advanced such that the distal end 135a of the pusher 130 abuts against the distal-end inner wall 112a of the drug solution container 110. For such a problem, the gasket 135 included in the pusher 130 according to the present embodiment is configured to be able to contract (be compressed) in a state of abutting against the distal-end inner wall 112a of the drug solution container 110, in an advancement direction (the longitudinal direction) of the pusher 130 when the pusher 130 advances. Therefore, the drug solution administration device 100 can further advance the pusher 130 by an amount of contraction (compression) of the gasket 135, even if the state shown in FIG. 11 is made to cause a situation where the advancement of the pusher 130 does not proceed. Therefore, in the drug solution administration device 100, the detection unit 150 can reliably detect the liquid supply completion.

A timing for ending the advancement of the pusher 130 and a timing for notifying the liquid supply completion may be set at any timing, for example, after a predetermined time has elapsed after the detection unit 150 detects the liquid supply completion. Further, the advancement of the pusher 130 may be continued until the drug solution administration device 100 is removed from the living body, or may be continued as it is after the liquid supply is completed.

(Administration Tool)

As shown in FIGS. 1 and 2, the administration tool 200 is configured to be connectable to the drug solution administration device 100.

The administration tool 200 has a connector 210, a needle tube or tubular needle (needle) 220 that is punctured into a living body, a puncture part (a cannula housing) 230, the tube 240, and a puncture assisting tool 250 that assists puncturing the needle tube 220 into the living body.

The connector 210 is configured to be connectable to the drug solution administration device 100 via a mounting part 215 fixed to the connector 210. The mounting part 215 can be connected to the drug solution administration device 100 by being externally fitted to the mounting part 115 (see FIG. 4) provided near the distal end 112 of the drug solution container 110 projecting to an outside of the housing 120.

Inside the mounting part 215, there is arranged a connecting needle part (not shown) capable of puncturing or configured to puncture/pierce a sealing member (not shown) arranged at the distal end portion of the drug solution container 110. The tube 240 communicates with the inner cavity 111a of the main body 111 of the drug solution container 110 via the connecting needle part.

Inside the puncture part 230, a flow path that connects or fluidly communicates the tube 240 and an inner cavity of the needle tube 220 is formed. A drug solution supplied to the puncture part 230 via the tube 240 is administered into the living body through the flow path formed inside the puncture part 230 and the needle tube 220.

The puncture assisting tool 250 is attached to the puncture part 230 at a time of liquid supply of the drug solution to the user. The puncture assisting tool 250 holds an introduction needle (an inner needle) 251. The introduction needle 251 passes through the needle tube 220 and projects from a distal end of the needle tube 220, with the puncture assisting tool 250 attached to the puncture part 230. By puncturing the living body with the needle tube 220 while the introduction needle 251 is positioned in the needle tube 220, the user can insert the needle tube 220 into a living body while preventing breakage or the like of the needle tube 220.

The puncture assisting tool 250 is removed from the puncture part 230 after the needle tube 220 is punctured into the living body. The introduction needle 251 is extracted from the inner cavity of the needle tube 220 when the puncture assisting tool 250 is removed from the puncture part 230.

After the needle tube 220 is punctured into the living body, the puncture assisting tool 250 is removed, and the puncture part 230 is left on the body surface H of the user with the needle tube 220 placed or positioned in the living body. In this state, as the pusher 130 of the drug solution administration device 100 advances in the drug solution container 110, the drug solution filled in the drug solution container 110 is supplied to the inner cavity (lumen) of the needle tube 220 via the tube 240 and the flow path of the puncture part 230.

The introduction needle 251 can be configured by, for example, a metal needle. Further, the needle tube 220 can be configured by, for example, a resin tubular member (a cannula).

The administration tool 200 is configured as a patch-type tool to be used by being attached to the body surface H of the user, similarly to the drug solution administration device 100. A contact surface (a bottom surface) 231 of the puncture part 230 of the administration tool 200 is provided with a sheet-shaped sticking part (not shown) that can be stuck to the body surface. In an initial state before attaching the administration tool 200 to the user, a releasable protective sheet is attached to a sticking surface of the sticking part.

As described above, the drug solution administration device 100 according to the present embodiment includes the drug solution container 110 filled with a drug solution and having the distal end opening at the distal end 112, the housing 120 that holds the drug solution container 110, the pusher 130 that pushes out the drug solution in the drug solution container 110, the drive mechanism 140 that advances the pusher 130 toward the distal end opening of the drug solution container 110, the detection unit 150 that detects the detection target part 134 of the pusher 130 and detects liquid supply completion of the drug solution based on a detection result, and the control unit 160 that controls an operation of the drive mechanism 140. The control unit 160 further advances the pusher 130 after the detection unit 150 detects the liquid supply completion.

According to the drug solution administration device 100 configured as described above, it is possible to prevent advancement of the pusher 130 from ending in a state where the drug solution remains in the drug solution container 110, since the pusher 130 is further advanced after liquid supply completion is detected by the detection unit 150. Therefore, it is possible to prevent liquid supply or injection of the drug solution from ending in a state where the drug solution remains in the drug solution container 110.

Further, in the drug solution administration device 100, the detection unit 150 is arranged so as to detect liquid supply completion before the distal end 135a of the pusher 130 abuts against the distal-end inner wall 112a of the drug solution container 110. Furthermore, the control unit 160 advances the pusher 130 by a distance equal to or more than a distance until the distal end 135a of the pusher 130 abuts against the distal-end inner wall 112a of the drug solution container 110 after the detection unit 150 detects the liquid supply completion. Therefore, the drug solution administration device 100 can more reliably prevent liquid supply of the drug solution from ending in a state where the drug solution remains in the drug solution container 110.

Further, in the drug solution administration device 100, the first distance (d1) from the distal end 135a of the pusher 130 to the detection target part 134 is shorter than the second distance (d2) from the distal-end inner wall 112a of the drug solution container 110 to the position where the detection unit 150 is arranged. Therefore, the detection unit 150 can reliably detect liquid supply completion before the distal end 135a of the pusher 130 abuts against the distal-end inner wall 112a of the drug solution container 110. Therefore, by further advancing the pusher 130 after detecting the liquid supply completion, it is possible to more reliably prevent liquid supply or injection of the drug solution from ending in a state where the drug solution remains in the drug solution container 110.

Further, in the drug solution administration device 100, the gasket 135 slidable with respect to the inner wall of the drug solution container 110 is arranged at the distal end 135a of the pusher 130. Further, when the pusher 130 advances, the gasket 135 can contract or compress in a state of abutting against the distal-end inner wall 112a of the drug solution container 110, in an advancement direction of the pusher 130. Therefore, the pusher 130 can be further advanced by an amount of contraction (compression) of the gasket 135, even in a state where the gasket 135 abuts against the distal-end inner wall 112a of the drug solution container 110 and a state where liquid supply completion is not detected by the detection unit 150. Therefore, the drug solution administration device 100 can more reliably detect the liquid supply completion.

Further, the control method for the drug solution administration device 100 according to the present embodiment is a control method for controlling movement of the pusher 130 in the drug solution administration device 100 including the drug solution container 110 filled with a drug solution and having the distal end opening at the distal end 112, the housing 120 that holds the drug solution container 110, the pusher 130 that pushes out the drug solution in the drug solution container 110, the drive mechanism 140 that advances the pusher 130 toward the distal end opening of the drug solution container 110, the detection unit 150 that detects the detection target part 134 of the pusher 130 and detects liquid supply completion of the drug solution based on a detection result, and the control unit 160 that controls an operation of the drive mechanism 140. In the control method, the control unit 160 further advances the pusher 130 after the detection unit 150 detects the liquid supply completion.

According to the above control method, since the pusher 130 is further advanced after liquid supply completion is detected by the detection unit 150, it is possible to prevent advancement of the pusher 130 from ending in a state where the drug solution remains in the drug solution container 110. Therefore, it is possible to prevent liquid supply of the drug solution from ending in a state where the drug solution remains in the drug solution container 110.

### Modified Example

The drug solution administration device 100 can also be configured to determine an administration state (state of administering the drug solution), for example, on the basis of a detection result of a rotation state of the motor 141 and on the basis of detection of liquid supply completion based on contact between the detection unit 150 and the detection target part 134 of the pusher 130. Specifically, the control unit 160 can be configured to determine the administration state of the drug solution administration device 100 with the following control flow.

When rotation stop of the motor 141 is detected by a rotation detection mechanism (not shown), the control unit 160 confirms detection of liquid supply completion based on contact between the detection target part 134 of the pusher 130 and the detection unit 150. When the motor 141 is stopped, the distal end 135a of the pusher 130 is in a state of abutting against the distal-end inner wall 112a of the drug solution container 110 (the state shown in FIG. 11). Therefore, after the detection unit 150 detects the liquid supply completion, the control unit 160 advances the pusher 130 until the rotation detection mechanism detects a stop of the rotation of the motor 141. Then, when the motor 141 is stopped, the control unit 160 determines that administration of the drug solution has ended normally, when confirming the liquid supply completion based on contact between the detection target part 134 of the pusher 130 and the detection unit 150 (when the pusher 130 and the detection unit 150 are in a contact state). Whereas, when the motor 141 is stopped, the control unit 160 determines that administration of the drug solution has not ended normally (that is, the administration of the drug solution has ended with the residual liquid remaining in the drug solution container 110), when confirming no detection of liquid supply completion based on contact between the detection target part 134 of the pusher 130 and the detection unit 150 (when the pusher 130 and the detection unit 150 are in a non-contact state). Thus, when the system determines that the motor 141 is stopped and that the detection target part 134 of the pusher 130 has contacted the detection unit 150, the system determines that administration of the drug solution has been normally performed and is completed. On the other hand, when the when the system determines the motor 141 is stopped but the detection target part 134 of the pusher 130 has not contacted the detection unit 150, the system determines that administration of the drug solution has not been normally performed. When the control unit 160 confirms that the administration of the drug solution has not been normally performed, the control unit 160 can notify that effect by the notification unit 180.

As described above, the drug solution administration device and the control method for the drug solution administration device disclosed here have been described through the embodiment explained above. However, the present invention is not limited to the particular details described above, but can be modified as appropriate within the scope of the claims.

For example, for the control unit, a specific control method, control procedure, and the like are not particularly limited as long as an operation is controlled to further advance the pusher after the detection unit detects liquid supply completion. Further, the housing may be configured such that each component member is fixed or held in the housing main body without including the chassis.

Further, a material, a shape, a size, arrangement, a connection/coupling structure between members, and the like of each member included in the drug solution administration device can be freely changed and replaced without limitation as long as the effects of the present invention are exhibited. Further, it is possible to appropriately add any component members and the like not particularly described in the specification, to the drug solution administration device.

The detailed description above describes embodiments of a drug solution administration device, a drug solution administration system and a control method representing examples of the inventive drug solution administration device, drug solution administration system and control method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A drug solution administration system comprising:
a housing;
a drug solution container filled with a drug solution and positioned in the housing, the drug solution container including a distal end;
a plunger positioned in the drug solution container so that the drug solution in the drug solution container is between the discharge port of the drug solution container and a distal end of the plunger, the plunger being movable relative to the drug solution container to advance toward the distal end of the drug solution container to discharge the drug solution out of the drug solution container through the distal end of the drug solution container, the plunger including a detection target part that moves together with the plunger;
a needle configured to puncture a living body, the needle being in fluid communication with the distal end of the drug solution container so that the drug solution discharged out of the drug solution container through the distal end of the drug solution container passes through the needle
a motor operatively connected to the plunger to advance the plunger toward the distal end of the drug solution container to discharge the drug solution out of the drug solution container through the distal end;
a sensor that detects the detection target part of the plunger as the plunger advances toward the discharge port of the drug solution container to identify a liquid supply completion state of the drug solution administration system, the sensor emitting a signal when the sensor detects the detection target part of the plunger; and
a control unit that receives the signal from the sensor when the sensor detects the detection target part of the plunger and that is operatively connected to the motor to control operation of the motor, the control unit being configured to operate the motor after the sensor detects the detection target part of the plunger so that there is further advancement of the plunger toward the distal end of the drug solution container after the sensor detects the detection target part of the plunger.

2. The drug solution administration system according to claim 1, wherein the needle is spaced from the distal end of the drug solution container.

3. The drug solution administration system according to claim 1, wherein the needle is connected to the distal end of the drug solution container by a tube.

4. The drug solution administration system according to claim 1, wherein the motor is connected to a battery.

5. The drug solution administration system according to claim 1, further comprising a gear reduction mechanism that transmits rotational driving force of the motor, and a feed screw connected to the gear reduction mechanism and the plunger to advance the plunger toward the distal end of the drug solution container.

6. The drug solution administration system according to claim 1, wherein the sensor detects the detection target part of the plunger before the distal end of the plunger abuts against a distal-end inner wall of the drug solution container, and after the sensor detects the detection target part of the plunger the control unit controls the operation of the motor so that the further advancement of the plunger includes advancing the plunger by a distance equal to or greater than a distance until the distal end of the plunger abuts against the distal-end inner wall of the drug solution container.

7. The drug solution administration system according to claim 6, wherein a first distance from the distal end of the pusher to the detection target part is shorter than a second distance from the distal-end inner wall of the drug solution container to the sensor before operating the motor.

8. The drug solution administration system according to claim 1, further comprising a gasket slidable with respect to an inner wall of the drug solution container, the gasket being positioned at the distal end of the plunger, the gasket being compressible so that when the plunger advances toward the distal end of the drug solution container and the gasket contacts the distal-end inner wall of the drug solution container the gasket compresses.

9. A drug solution administration device comprising:
a drug solution container filled with a drug solution and having a distal end opening at a distal end of the drug solution container;
a housing in which the drug solution container is positioned;
a plunger that pushes out the drug solution in the drug solution container so that the drug solution is discharged out of the distal end opening at the distal end of the drug solution container, the plunger including a detection target part;
a drive mechanism operatively connected to the plunger to advance the plunger toward the distal end opening of the drug solution container;
a detection unit that detects the detection target part of the plunger and detects liquid supply completion of the drug solution when the detection unit detects the detection target part of the plunger; and
a control unit that is operatively connected to the drive mechanism and that controls operation of the drive mechanism based on detection results received from the detection unit, the control unit being configured to operate the drive mechanism to further advance the plunger toward the distal end opening of the drug solution container after the detection unit detects the liquid supply completion.

10. The drug solution administration device according to claim 9, wherein
the detection unit is configured to detect the liquid supply completion before a distal end of the plunger abuts against a distal-end inner wall of the drug solution container, and
after the detection unit detects the liquid supply completion, the control unit advances the plunger by a distance equal to or greater than a distance until the distal end of the plunger abuts against the distal-end inner wall of the drug solution container.

11. The drug solution administration device according to claim 10, wherein a first distance from the distal end of the plunger to the detection target part is shorter than a second distance from the distal-end inner wall of the drug solution container to the detection unit.

12. The drug solution administration device according to claim 9, wherein
a gasket slidable with respect to an inner wall of the drug solution container is arranged at a distal end of the plunger, and
when the plunger advances toward the distal end opening of the drug solution container, the gasket is compressible when a distal end of the gasket abuts against the distal-end inner wall of the drug solution container.

13. The drug solution administration device according to claim 12, wherein a first distance from a distal end of the plunger to the detection target part is shorter than a second distance from the distal-end inner wall of the drug solution container to the detection unit.

14. The drug solution administration device according to claim 9, wherein a first distance from a distal end of the plunger to the detection target part is shorter than a second distance from the distal-end inner wall of the drug solution container to the detection unit.

15. The drug solution administration device according to claim 9, wherein the drive mechanism includes a motor connected to a power supply unit so that the motor produces a rotational driving force, a gear reduction mechanism that transmits the rotational driving force of the motor, and a feed screw connected to the gear reduction mechanism to advance the plunger toward the distal end opening of the drug solution container.

16. A control method for controlling movement of a plunger positioned in a drug solution container that is filled with a drug solution so that the drug solution is between the plunger and a distal end of the drug solution container, the drug solution container and the plunger being positioned in a housing, the drug solution container including a distal end opening at the distal end of the drug solution container, the plunger including a detection target part that moves together with the plunger, and further comprising a detection unit that detects the detection target part of the plunger, the control method comprising:
advancing the plunger inside the drug solution container toward the distal end opening of the drug solution container to discharge the drug solution out of the drug solution container through the distal end opening of the drug solution container;
detecting the detection target part through operation of the detection unit to determine that a liquid supply completion state has been reached; and
further advancing the plunger toward the distal end opening of the drug solution container after the detecting of the detection target part through operation of the detection unit.

17. The control method according to claim 16, wherein the plunger includes a distal end at which is located a compressible gasket that is slidable with respect to an inner wall of the drug solution container as the plunger is advanced toward the distal end opening of the drug solution container, and wherein the further advancing of the plunger toward the distal end opening of the drug solution container includes further advancing the plunger toward the distal end opening of the drug solution container to abut a distal end of the compressible gasket against the distal-end inner wall of the drug solution container and to compress the compressible gasket.

18. The control method according to claim 16, wherein the advancing of the plunger inside the drug solution container toward the distal end opening of the drug solution container occurs while the distal end opening of the drug solution container is in fluid communication with a needle and while the needle is in a living body so that the drug solution is delivered to the body.

19. The control method according to claim 16, wherein the advancing of the plunger inside the drug solution container occurs through operation of a motor positioned in the housing.
